(19) United States
(12) Patent Application Publication
(10) Pub. No.: US 2018/0043154 A1
(43) Pub. Date: Feb. 15, 2018

(54) MUSCLE TRAINING DEVICE

(71) Applicant: SMK Corporation, Tokyo (JP)

(72) Inventors: Koichiro EJIRI, Kanagawa (JP); Haruhiko KONDO, Kanagawa (JP)

(21) Appl. No.: 15/593,336

(22) Filed: May 12, 2017

(30) Foreign Application Priority Data
Aug. 10, 2016 (JP) 2016-158006

(51) Int. Cl. A61N 1/36; A61N 1/04

(52) U.S. Cl. CPC A61N 1/36003; A61N 1/0452; A61N 1/0476; A61N 1/36014; A61N 1/0492

(57) ABSTRACT

A muscle training device includes an insulating sheet configured such that electrodes are exposed on an inner surface closely contacting a body surface above a muscle, a stimulation signal output unit configured to output an electrical stimulation signal to a stimulation electrode which is at least any of the electrodes, a myoelectric signal detection unit configured to detect, while output of the electrical stimulation signal is stopped, a muscle action potential shown at a myoelectric detection electrode which is at least any of the electrodes, and a muscle condition determination unit configured to determine the state of action of the muscle from the muscle action potential of the myoelectric detection electrode. The stimulation electrode to which the electrical stimulation signal is output and/or the method for outputting the electrical stimulation signal are/is adjusted according to the state of action of the muscle determined by the muscle condition determination unit.

### CROSS REFERENCE TO RELATED APPLICATION

The contents of the following Japanese patent application are incorporated herein by reference.

Japanese Patent Application No. 2016-158006 filed on Aug. 10, 2016.

### FIELD

The present invention relates to a muscle training device utilizing electrical muscle stimulation (EMS) for applying an electrical stimulation signal to a body surface above a muscle to expand and contract the muscle. More specifically, the present invention relates to a muscle training device configured to apply an optimal electrical stimulation signal according to a muscle condition to effectively train a muscle.

### BACKGROUND

A muscle training device configured to apply an electrical stimulation signal to a muscle to expand and contract the muscle for training for the purpose of muscle strength enhancement and dieting has been typically known (JP-A-2005-348859). FIGS. 8 and 9 illustrate a muscle training device 100 described in JP-A-2005-348859. For training of an abdominal muscle, four stimulation electrodes 101a, 101b, 101c, 101d closely contact a body surface of the abdomen.

An electrical stimulation output part 102 is configured to generate an electrical stimulation signal output to each of the four stimulation electrodes 101a, 101b, 101c, 101d based on an instruction from a controller 103. For example, the electrical stimulation output part 102 outputs an electrical stimulation signal of 1000 Hz to between the stimulation electrodes 101a, 101c arranged on one diagonal line and included in the four stimulation electrodes 101a, 101b, 101c, 101d, and outputs an electrical stimulation signal of 900 Hz to between the stimulation electrodes 101b, 101d arranged on the other diagonal line. This generates an interference wave of 10 Hz between adjacent ones of the stimulation electrodes 101. Accordingly, an abdominal muscle of such a portion is strengthened by expansion and contraction thereof

In a storage part 104 connected to the controller 103, correlation data between the frequency and current value of the electrical stimulation signal is stored. The controller 103 instructs the electrical stimulation output part 102 to generate the electrical stimulation signal according to the correlation data stored in the storage part 104. Moreover, a display 105 displays the degree of stimulation by the electrical stimulation signal output to each of the stimulation electrodes 101a, 101b, 101c, 101d. While viewing such a displayed indication, a user operates, when feeling painful due to an electrical stimulation, an operation part 106 to lower an output level such as the current value of the electrical stimulation signal generated by the electrical stimulation output part 102 via the controller 103.

Moreover, a muscle training device 110 configured to apply an electrical stimulation signal to a muscle for training and to obtain a muscle composition in an objective manner has been also known (JP-A-2016-49241). As illustrated in FIG. 10, the muscle training device 110 is configured such that a pair of stimulation electrodes 111a, 111b and a photoelectric sensor 112 between the stimulation electrodes 111a, 111b are arranged in close contact with a body surface above a muscle to be trained. The photoelectric sensor 112 can measure the concentration of oxygen in the muscle under the body surface to which the photoelectric sensor 112 closely contact. The concentration of oxygen in the muscle changes in response to an electrical stimulation applied from the pair of stimulation electrodes 111a, 111b to the muscle. Thus, information on a muscle composition (e.g., a ratio between a slow muscle and a fast muscle) is obtained from a relationship between the strength of the electrical stimulation and the measured oxygen concentration.

Thus, the muscle training device 110 is operable between the following two types of modes: a training mode in which the electrical stimulation signal is output from a device body 113 to between the stimulation electrodes 111a, 111b, and a load due to the electrical stimulation is provided to the muscle under the body surface to perform training; and a measurement mode in which while the strength of electrical stimulation of the muscle by the pair of stimulation electrodes 111a, 111b is being changed, the concentration of oxygen in the muscle is measured by the photoelectric sensor 112 to obtain the information on the muscle composition in the device body 113. For example, according to the ratio between the slow muscle and the fast muscle in the measurement mode, the strength of the electrical stimulation provided to the muscle in the training mode is adjusted.

### SUMMARY

### Technical Problem

In the typical muscle training device 100 utilizing the electrical muscle stimulation EMS, the stimulation electrodes 101 closely contact the body surface above the muscle to be trained. However, the position of the muscle covered with the body surface cannot be accurately determined. For this reason, the position of the muscle and the electrical stimulation position shift from each other, and the electrical stimulation cannot be effectively applied to the muscle. Particularly for outputting an electrical stimulation signal to between stimulation electrodes as a pair of positive and negative electrodes to effectively expand and contract a muscle, the positive and negative electrodes need to closely contact a body surface along the longitudinal direction of the muscle. Thus, the device needs to rely on a skilled user's experience to determine such a close contact position.

Moreover, an action state such as the degree of fatigue of the muscle to be trained cannot be determined in an objective manner, and for this reason, the strength of the electrical stimulation and a stimulation time cannot be optimally adjusted according to the state of action of the muscle.

According to the muscle training device 110, the strength of electrical stimulation to the muscle can be optimally set according to the composition of the muscle to be trained. However, for evaluation of the muscle composition, the photoelectric sensor 112 needs to be separately prepared and to closely contact the body surface. In addition, the photoelectric sensor 112 and the pair of stimulation electrodes 111a, 111b closely contact different positions of the body surface. Thus, unlike information on the composition of the muscle to which the electrical stimulation is actually provided, an optimal strength of electrical stimulation might not be able to be applied based on the muscle composition information obtained from the photoelectric sensor 112.

Further, the photoelectric sensor 112 only obtains the muscle composition information from the relationship between the electrical stimulation strength and the muscle oxygen concentration. For this reason, the degree of fatigue of the muscle due to the electrical stimulation and positional shift between the electrical stimulation position of the stimulation electrode and the muscle position cannot be determined, for example. Thus, the electrical stimulation cannot be applied to the muscle in an optimal manner considering the above.

The present invention has been made in view of the above-described typical problems, and intends to provide a muscle training device configured to provide an electrical stimulation to a muscle in an optimal manner according to the state of action of the muscle without the need for closely contacting a separate photoelectric sensor to a body surface above the muscle.

Moreover, the present invention intends to provide a muscle training device being able to closely contact a stimulation electrode to a proper position of a body surface to effectively apply an electrical stimulation to a muscle.

Further, the present invention intends to provide a muscle training device configured to provide an electrical stimulation to a muscle with an optimal strength for an optimal time according to the degree of fatigue of the muscle.

### Solution to Problem

For solving the above-described problems, the muscle training device according to a first aspect is a muscle training device for applying a first electrical stimulation signal for training to a body surface above a muscle to expand and contract the muscle by the first electrical stimulation signal. Such a muscle training device includes an insulating sheet configured such that a plurality of electrodes are exposed on an inner surface closely contacting the body surface above the muscle, a first stimulation signal output unit configured to output the first electrical stimulation signal to a stimulation electrode which is at least any of the plurality of electrodes, a myoelectric signal detection unit configured to detect, while output of the first electrical stimulation signal is stopped, a muscle action potential shown at a myoelectric detection electrode which is at least any of the plurality of electrodes, and a muscle condition determination unit configured to determine the state of action of the muscle from the muscle action potential of the myoelectric detection electrode.

The plurality of electrodes provided on the insulating sheet can be used in combination as the stimulation electrode for training and the myoelectric detection electrode for determination of the muscle action state.

In the muscle training device according to a second aspect, the stimulation electrode to which the first electrical stimulation signal is output and/or the method for outputting the first electrical stimulation signal are/is adjusted according to the state of action of the muscle determined by the muscle condition determination unit.

The stimulation electrode to which the first electrical stimulation signal is output and/or the method for outputting the first electrical stimulation signal are/is adjusted according to the state of action of the muscle without the need for providing a separate myoelectric detection electrode in addition to the stimulation electrode for training.

In the muscle training device according to a third aspect, the plurality of electrodes dispersedly exposed on a two-dimensional plane along the inner surface of the insulating sheet serve as myoelectric detection electrodes, and the stimulation electrode to which the first electrical stimulation signal is output is selected from ones of the plurality of myoelectric detection electrodes from which a relatively-high muscle action potential has been detected.

Since the myoelectric detection electrodes are dispersedly exposed on the two-dimensional plane along the inner surface of the insulating sheet, any of the myoelectric detection electrodes closely contacts the body surface near the muscle. Since the muscle generates the muscle action potential in association with expansion and contraction thereof, a relatively-high muscle action potential is detected from the myoelectric detection electrode closely contacting the body surface near the muscle. Such a myoelectric detection electrode serves as the stimulation electrode, and therefore, the first electrical stimulation signal is output to the body surface near the muscle regardless of the bonded position of the insulating sheet to the body surface.

The muscle action potential is a potential formed in such a manner that action potentials generated at a plurality of muscle fibers overlap with each other, and is transmitted at 3 to 5 m/sec along the longitudinal direction of the muscle formed of the plurality of muscle fibers. Such a transmission velocity decreases as fatigue of the muscle increases. Any of the myoelectric detection electrodes from which a relatively-high muscle action potential has been detected closely contacts the body surface along the muscle. The transmission velocity of the muscle action potential is obtained from a time difference in detection of the muscle action potential by each myoelectric detection electrode and an already-provided distance between the myoelectric detection electrodes. Then, the muscle condition determination unit determines the degree of fatigue of the muscle in a quantitative manner.

In the muscle training device according to a fourth aspect, the plurality of electrodes dispersedly exposed at positions in a grid pattern on the inner surface of the insulating sheet serve as the myoelectric detection electrodes.

Since the plurality of myoelectric detection electrodes are dispersedly exposed at the positions in the grid pattern, it is ensured that any of the myoelectric detection electrodes closely contacts the body surface along the longitudinal direction of the muscle and serves as the myoelectric detection electrode from which a relatively-high muscle action potential is detected.

In the muscle training device according to a fifth aspect, the plurality of electrodes dispersedly exposed at positions in a concentric pattern on the inner surface of the insulating sheet serve as the myoelectric detection electrodes.

The plurality of electrodes are dispersedly exposed on the two-dimensional plane of the bottom surface. Thus, even when the body surface near the muscle to be trained is curved, at least any of the electrodes closely contacts the body surface near the muscle.

In the muscle training device according to a sixth aspect, a stimulation sub-electrode including at least one electrode closely contacts the body surface along the muscle in the vicinity of the body surface to which the insulating sheet closely contacts. The first electrical stimulation signal output unit causes one of the stimulation electrode or the stimulation sub-electrode on the insulating sheet to serve as a positive electrode and causes the other one of the stimulation electrode or the stimulation sub-electrode on the insulating sheet to serve as a negative electrode, thereby outputting the first electrical stimulation signal to between the positive and negative electrodes.

The stimulation electrode and the stimulation sub-electrode on the insulating sheet can closely contact distant positions of the body surface along the muscle, and therefore, the first electrical stimulation signal flows between the stimulation electrode and the stimulation sub-electrode along the direction of the muscle fibers of the muscle.

In the muscle training device according to a seventh aspect, a pair of insulating sheets closely contact two body surface portions along the muscle, and any of a plurality of electrodes exposed on an inner surface of one of the insulating sheets serves as a stimulation sub-electrode.

The electrodes exposed on the inner surface of each insulating sheet serve as the myoelectric detection electrodes, and the state of action of the muscle at each position of the body surface closely contacting the insulating sheet can be determined.

Since any of the plurality of electrodes exposed on the inner surface of one of the insulating sheets serves as the stimulation sub-electrode, the first electrical stimulation signal flows along the direction of the muscle fibers of the muscle between the stimulation electrode and the sub-electrode apart from each other on the body surface above the muscle.

The muscle training device according to an eighth aspect further includes a second stimulation signal output unit configured to apply, while output of the first electrical stimulation signal is stopped, a second electrical stimulation signal for generating an induced muscle action potential to the body surface along the muscle in the vicinity of the body surface to which the insulating sheet closely contacts. The myoelectric signal detection unit detects the induced muscle action potential shown at the myoelectric detection electrode by application of the second electrical stimulation signal.

The M-wave of the induced muscle action potential induced by the second electrical stimulation signal is transmitted along the direction of the muscle fibers of the muscle from the body surface to which the second electrical stimulation signal is applied, and is detected by each of the myoelectric detection electrodes closely contacting different positions of the body surface along the muscle.

The transmission velocity of the induced muscle action potential for evaluation of the degree of fatigue of the muscle in a quantitative manner is obtained from a time at which the M-wave reaches each myoelectric detection electrode.

According to the first aspect of the invention, the state of action of the muscle to be trained can be determined without the need for bonding a separate device such as a photoelectric sensor to the body surface.

According to the second aspect of the invention, stimulation strength and a stimulation time by the first electrical stimulation signal can be adjusted such that the muscle is trained in an optimal manner according to the state of action of the muscle determined by the muscle condition determination unit.

According to the third aspect of the invention, the first electrical stimulation signal can be applied to the body surface above the muscle without time and effort for closely contacting the stimulation electrode to the body surface along the muscle.

Moreover, the myoelectric detection electrode from which a relatively-high muscle action potential has been detected closely contacts the vicinity of the muscle. Thus, the relative transmission velocity of the muscle action potential indicating the degree of fatigue of the muscle is obtained from the time difference in detection of the muscle action potential by the myoelectric detection electrode, and the strength and output time of the first electrical stimulation signal can be adjusted according to the degree of fatigue of the muscle determined by the muscle condition determination unit.

According to the fourth aspect of the invention, the first electrical stimulation signal can be reliably output to the stimulation electrode closely contacting the body surface near the muscle regardless of the direction of the insulating sheet bonded to the body surface.

According to the fifth aspect of the invention, at least any of the electrodes closely contacts the body surface near the muscle. Thus, even when the body surface near the muscle to be trained is curved, a relatively-high muscle action potential can be detected from the closely-contacting electrode, and a training stimulation signal can be effectively applied to the muscle.

According to the sixth aspect of the invention, even when the muscle has such a size that the insulating sheet cannot cover the muscle, the first electrical stimulation signal flows in the direction of the muscle fibers of the muscle such that the muscle expands and contracts.

According to the seventh aspect of the invention, the state of action of the muscle at each position closely contacting the pair of insulating sheets can be determined. Moreover, the first electrical stimulation signal flows in the direction of the muscle fibers of the muscle through the muscle having such a size that the single insulating sheet cannot cover the muscle, and therefore, the muscle can expand and contract.

According to the eighth aspect of the invention, the induced muscle action potential is generated at the muscle by the second electrical stimulation signal. Thus, the muscle action potential can be reliably detected by the myoelectric detection electrode distinctively from noise of an electric signal of an instruction from the brain and an electric signal generated from a muscle due to an exercise load.

Moreover, the transmission velocity of the induced muscle action potential induced by the second electrical stimulation signal only reflects a change in a peripheral state, and delays due to fatigue of the muscle. Thus, the muscle can be trained by output of the first electrical stimulation signal in an optimal manner according to fatigue of the muscle for accurate quantitative evaluation.

### BRIEF DESCRIPTION OF DRAWINGS

FIG. 1 is a reference diagram of a use state of a muscle training device 1 of a first embodiment of the invention of the present application;

FIG. 2 is a block diagram of the muscle training device 1;

FIG. 3 is a reference diagram of a use state of a muscle training device 20 of a second embodiment;

FIG. 4 is a reference diagram of a use state of a muscle training device 30 of a third embodiment;

FIG. 5 is a block diagram of a muscle training device 40 of a fourth embodiment;

FIG. 6 is a reference diagram of a use state of the muscle training device 40;

FIG. 7 is a bottom view of an insulating sheet 5;

FIG. 8 is a block diagram of a typical muscle training device 100;

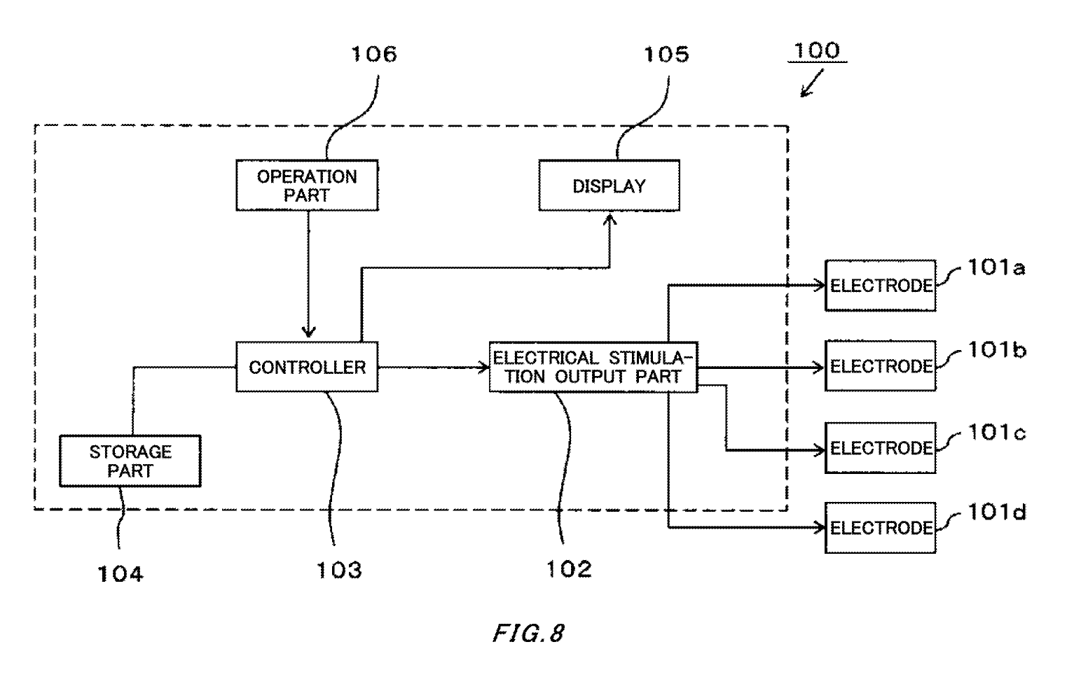

FIG. 9 is a view of a use state of the muscle training device 100; and

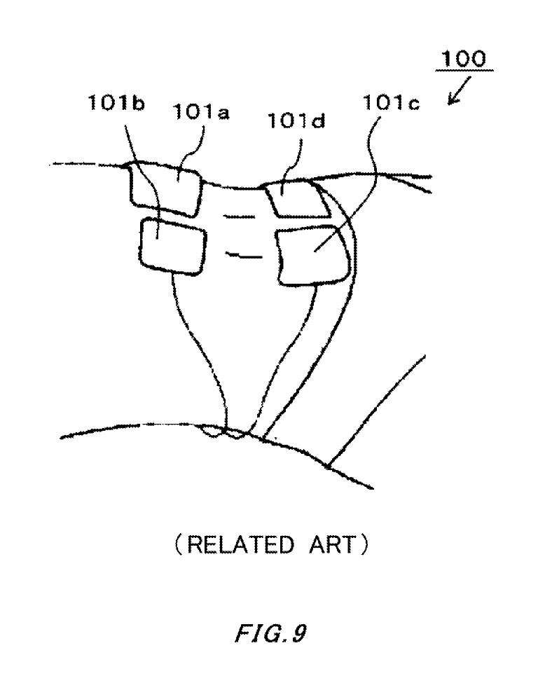

FIG. 10 is a view of a configuration of a typical muscle training device 110.

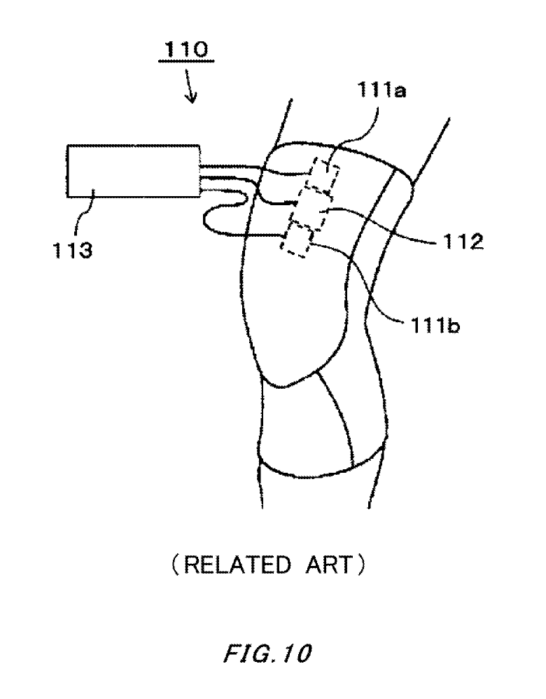

### DESCRIPTION OF EMBODIMENTS

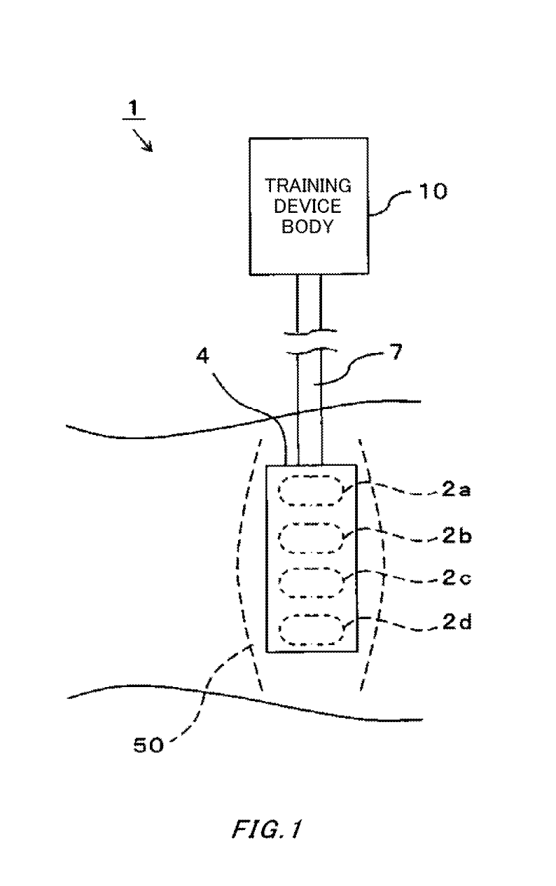

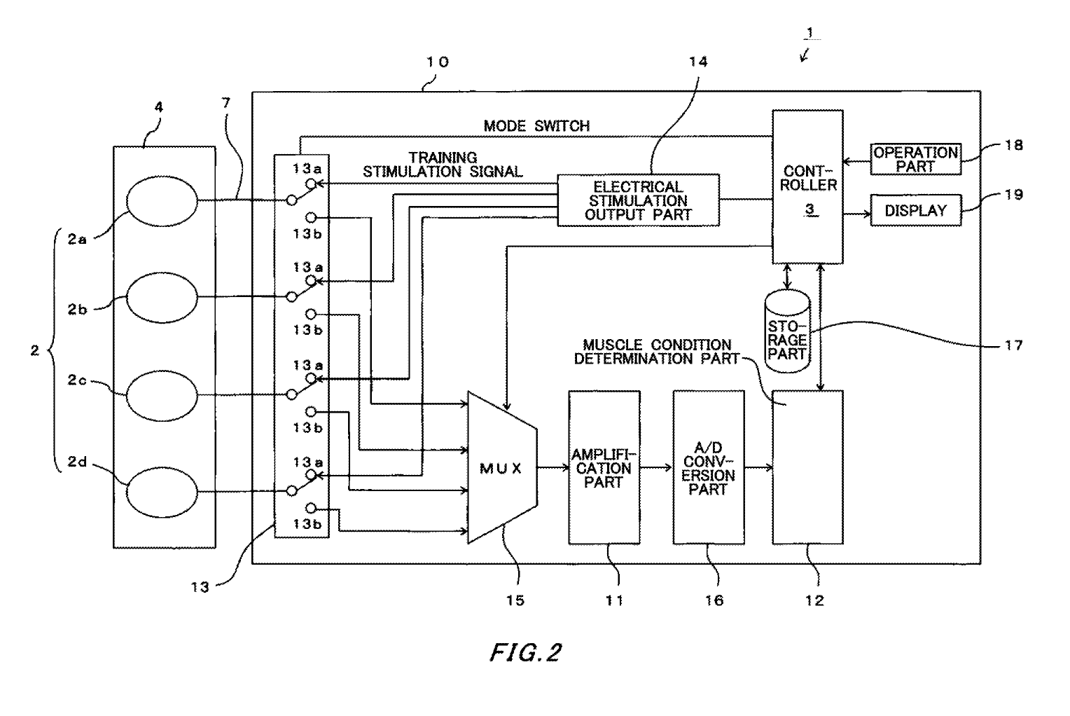

A muscle training device 1 of a first embodiment of the present invention will be described below with reference to FIGS. 1 and 2. As illustrated in FIG. 1, the muscle training device 1 is a device configured to expand and contract a muscle 50 by application of an electrical stimulation signal to the muscle 50, thereby training the muscle 50. In the present specification, training of the muscle 50 means, regardless of purposes, electrical muscle stimulation (EMS) for expanding and contracting the muscle 50 by application of a later-described training stimulation signal as an electrical stimulation signal to a body surface above the muscle 50. Thus, the muscle training device 1 may be any of the following devices configured to expand and contract the muscle 50 for various purposes: a muscle training device for the purpose of dieting for surrounding fat combustion by expansion and contraction of a muscle 50; a muscle training device for the purpose of enhancing muscle strength; and a medical muscle training device configured to regenerate a damaged muscle 50.

The muscle training device 1 includes an insulating sheet 4 to which four electrodes 2a, 2b, 2c, 2d for providing an electrical stimulation to the muscle 50 under the body surface are attached in an insulated state, and a training device body 10 configured to output, to any of the four electrodes 2a, 2b, 2c, 2d, a training stimulation signal as an electrical stimulation signal for training via a connection cable 7.

The outer shape of the insulating sheet 4 is defined by an elongated band-shaped flexible printed circuit board (FPC) so that the insulating sheet 4 can be positioned on the body surface along a muscle fiber direction of the muscle 50 to be trained. The four electrodes 2a, 2b, 2c, 2d are, in the insulated state, formed integrally with the flexible insulating sheet 4 made of PET etc. The four electrodes 2a, 2b, 2c, 2d are exposed at regular intervals on a bottom surface of the insulating sheet 4, i.e., an inner surface of the insulating sheet 4 facing the body surface, and are each connected to a corresponding common terminal of a later-described switch 13 via a wiring pattern of the FPC and the connection cable 7. A not-shown double-faced tape is bonded to the substantially entire bottom surface of the insulating sheet 4 other than the portion where the four electrodes 2a, 2b, 2c, 2d are exposed. An adhesive layer exposed by detachment of release paper of the double-faced tape is bonded to the body surface above the muscle 50 to be trained, and in this manner, the insulating sheet 4 is positioned. As described above, the four electrodes 2a, 2b, 2c, 2d closely contact the body surface facing these electrodes.

As illustrated in FIG. 2, the training device body 10 includes the switch 13 configured to switch, according to a mode switch signal output from a controller 3, each common terminal between a first selection terminal 13a connected to an output of an electrical stimulation output part 14 and a second selection terminal 13b connected to an input of a multiplexer 15, each common terminal being connected to a corresponding one of the four electrodes 2a, 2b, 2c, 2d. When receiving the mode switch signal for selecting a particular electrode 2 to make transition to a training mode, the switch 13 switchably connects a common terminal connected to such an electrode 2 to the first selection terminal 13a, and the selected electrode 2 serves as a stimulation electrode to which the training stimulation signal is output from the electrical stimulation output part 14. For example, when the electrodes 2a, 2b are selected by the mode switch signal for making transition to the training mode, the common terminals connected respectively to the electrodes 2a, 2b are each switched to the first selection terminal 13a, and then, the training stimulation signal is output to each of the electrodes 2a, 2b. When the mode switch signal for selecting a particular electrode 2 to make transition to a muscle condition determination mode is output from the controller 3 to the switch 13, a common terminal connected to the selected electrode 2 is switched to the second selection terminal 13b, and such an electrode 2 serves as a myoelectric detection electrode configured to detect a muscle action potential.

In response to a control signal input from the controller 3, the multiplexer 15 alternately connects ones of the four types of second selection terminals 13b to an amplification part 11 connected to a later stage of the multiplexer 15, the ones of the second selection terminals 13b being connected respectively to the electrodes (the myoelectric detection electrodes) 2 selected by the mode switch signal for making transition to the muscle condition determination mode. Then, the multiplexer 15 outputs a muscle action potential of a myoelectric signal detected at each myoelectric detection electrode 2 to a muscle condition determination part 12 via the amplification part 11 and an A/D conversion part 16. That is, the muscle action potential of the myoelectric signal detected from the myoelectric detection electrode 2 closely contacting the body surface is a minute value. For this reason, after amplification at the amplification part 11, such a potential is converted into a digital value at the A/D conversion part 16 so that the muscle condition determination part 12 can make determination.

The muscle condition determination part 12 is configured to determine, from the level (the muscle action potential) and amplitude of the myoelectric signal detected by each myoelectric detection electrode 2, the position of the muscle 50 to be trained under the body surface and the state of fatigue of the muscle 50 due to training.

The position of the muscle 50 to be trained is estimated from the position of the body surface closely contacting the myoelectric detection electrode 2 from which a relatively-high muscle action potential has been detected. That is, among muscles, a skeletal muscle being able to voluntarily act under the control of brain nerves generates, in response to a stimulation from motor nerves, the muscle action potential at a plurality of muscle fibers forming the muscle, and then, contracts to generate tension. Thus, in the case where the muscle 50 to be trained is the skeletal muscle, a high muscle action potential is detected from the myoelectric detection electrode 2 closely contacting the body surface near the muscle 50, and the muscle condition determination part 12 estimates from many myoelectric detection electrodes 2 that the muscle 50 is present near the myoelectric detection electrode 2 from which a relatively-high muscle action potential has been detected.

When fatigue of the muscle 50 increases due to training, the number of mobilization activities and a firing frequency per motor unit for making up for contractility of the muscle 50 increase, and the amplitude of the myoelectric signal increases. Thus, a change in the amplitude of the myoelectric signal detected from the myoelectric detection electrode 2 having estimated as closely contacting the vicinity of the muscle 50 in the above-described manner is monitored, and the degree of fatigue of the muscle 50 is determined from an amplitude increase.

The velocity of transmission of the muscle action potential transmitted along the longitudinal direction (the muscle fiber direction) of the muscle 50 becomes lower as fatigue of the muscle 50 increases. Thus, the transmission velocity along the muscle 50 may be detected from a time difference in detection of the myoelectric signals having the same waveform by the plurality of myoelectric detection electrodes 2 closely contacting the vicinity of the muscle 50, and in this manner, the degree of fatigue of the muscle 50 may be determined.

The muscle condition determination part 12 is connected to the controller 3 to output a determination result of the muscle 50 by the muscle condition determination part 12 to the controller 3, and the controller 3 inputs, to the muscle condition determination part 12, information on the position of each myoelectric detection electrode 2 closely contacting the vicinity of the muscle 50 for determination of the degree of fatigue of the muscle 50 by the muscle condition determination part 12.

The controller 3 operates in any of the training mode and the muscle condition determination mode. The controller 3 is connected to a storage part 17 configured to store the determination result of the muscle condition determination part 12 and a series of operation program of the controller 3, an operation part 18 allowing a user to start the muscle training device 1 and to perform input operation for stopping operation, a display 19 configured to display, e.g., the progress of training and the degree of fatigue detected by the muscle condition determination part 12, and the electrical stimulation output part 14. When receiving a start-up instruction from the operation part 18, the controller 3 performs sequence control of each part of the muscle training device 1 such that the muscle training device 1 executes a series of operation from the muscle condition determination mode to the training mode.

Under the control of the controller 3 in the training mode, the electrical stimulation output part 14 generates the training stimulation signal to output the generated training stimulation signal to each first selection terminal 13a of the switch 13. The training stimulation signal has an AC signal waveform with a current value of about 10 to 80 mA providing no feeling of electrical stimulation to the user and with a frequency of 10 to 30 Hz, for example. The waveform and frequency of the training stimulation signal may vary according to the progress of the training mode to obtain an optimal training effect.

The method for training the skeletal muscle 50 by using the muscle training device 1 will be described below together with advantageous effects of the muscle training device 1. In the case where the user trains the skeletal muscle 50, the adhesive layer exposed by detachment of the release paper from the bottom surface of the insulating sheet 4 is bonded to the body surface in the vicinity of the muscle 50 to be trained, and in this manner, the four electrodes 2a, 2b, 2c, 2d closely contact the body surface. Subsequently, input operation for starting the muscle training device 1 is performed for the operation part 18.

After the muscle training device 1 has been started, the controller 3 first operates in the muscle condition determination mode, and then, outputs, to the switch 13, the mode switch signal for selecting all of the four electrodes 2a, 2b, 2c, 2d to make transition to the muscle condition determination mode. When receiving such a mode switch signal, the switch 13 switchably connects, to the second selection terminal 13b, each common terminal connected to a corresponding one of the four selected electrodes 2a, 2b, 2c, 2d. Then, each of the electrodes 2a, 2b, 2c, 2d serves as the myoelectric detection electrode configured to detect the muscle action potential.

Subsequently, when the user voluntarily expands and contracts the skeletal muscle 50, the muscle action potential is generated at the plurality of muscle fibers forming the muscle 50, and a relatively-high muscle action potential is detected from each myoelectric detection electrode 2 closely contacting the vicinity of these muscle fibers. The controller 3 outputs, to the switch 13, the mode switch signal for making transition to the above-described muscle condition determination mode, and serially connects, to the amplification part 11, the second selection terminals 13b connected respectively to the electrodes 2a, 2b, 2c, 2d selected by such a mode switch signal. Thus, after the muscle action potential detected from each of the electrodes 2a, 2b, 2c, 2d has been amplified in the amplification part 11, the resultant is converted into the digital value in the A/D conversion part 16, and then, is output to the muscle condition determination part 12. Then, relative comparison is made for the resultant. The muscle action potential detected from each of the myoelectric detection electrodes 2a, 2b, 2c is a relatively-higher muscle action potential than that of the myoelectric detection electrode 2d, supposing that the myoelectric detection electrodes 2a, 2b, 2c closely contact the vicinity of the muscle 50 and that the myoelectric detection electrode 2d closely contacts the body surface apart from the muscle 50. Thus, the muscle condition determination part 12 estimates, from such a comparison result, that the muscle 50 is along the electrodes 2a, 2b, 2c under the body surface. Then, the determination result including the amplitude of the myoelectric signal detected from each of the myoelectric detection electrodes 2a, 2b, 2c is output to the controller 3. In comparison of the muscle action potential detected from each myoelectric detection electrode 2, a value obtained by subtracting an abnormal value from a muscle action potential detected multiple times may be targeted for comparison, or the myoelectric detection electrode 2 having detected a muscle action potential equal to or higher than a predetermined potential may serve as the myoelectric detection electrode 2 having detected a relatively-high muscle action potential.

After the controller 3 has output, to the switch 13, the mode switch signal for making transition to the muscle condition determination mode, when the determination result of the position of the muscle 50 is input from the muscle condition determination part 12, such a determination result is primarily stored in the storage part 17, and the electrodes 2a, 2b, 2c estimated from the determination result as closely contacting the vicinity of the muscle 50 are selected as the stimulation electrodes. Then, the controller 3 outputs, to the switch 13, the mode switch signal for selecting the stimulation electrodes 2a, 2b, 2c to make transition to the training mode. After or before this point, the controller 3 controls the electrical stimulation output part 14 to generate and output the training stimulation signal.

The switch 13 having received the mode switch signal switchably connects, to the first selection terminal 13a, each common terminal connected to a corresponding one of the selected electrodes 2a, 2b, 2c, and the electrodes 2a, 2b, 2c serve as the stimulation electrodes 2a, 2b, 2c to which the training stimulation signal is output from the electrical stimulation output part 14. Thus, the training stimulation signal is output to the electrodes 2 closely contacting the vicinity of the muscle 50 so that the electrical stimulation can be effectively applied to the muscle 50 without the need for considering such bonded position and orientation of the insulating sheet 4 that each electrode 2 closely contacts the vicinity of the muscle 50 to be trained.

Note that all of the myoelectric detection electrodes 2 having detected a relatively-high muscle action potential in the muscle condition determination mode do not necessarily serve as the stimulation electrodes 2 to which the training stimulation signal is output. Of the myoelectric detection electrodes 2a, 2b, 2c having detected a high muscle action potential, one of the myoelectric detection electrodes 2a, 2c closely contacting distant positions may serve as a positive electrode, and the other one of the myoelectric detection electrodes 2a, 2c may serve as a negative electrode. The training stimulation signal may be output to between these electrodes, for example.

The controller 3 may terminate the training mode after a predetermined training time has been elapsed. However, in the present embodiment, the training mode is temporarily switched to the muscle condition determination mode in the process of the training mode, and then, the state of fatigue of the muscle 50 due to training is checked. Such mode switching is performed as follows: the indication of transition to the muscle condition determination mode is displayed on the display 19, and information on the electrodes 2a, 2b, 2c estimated from the determination result stored in the storage part 17 as closely contacting the vicinity of the muscle 50 is read; and then, the mode switch signal for selecting these electrodes 2a, 2b, 2c to make transition to the muscle condition determination mode is output to the switch 13.

When the user having checked the indication on the display 19 voluntarily expands and contracts the muscle 50, the muscle action potential is detected from each of the myoelectric detection electrodes 2a, 2b, 2c near such a muscle. After each potential value has been amplified in the amplification part 11, the resultant is converted into the digital value in the A/D conversion part 16, and the digital value is input to the muscle condition determination part 12. The muscle condition determination part 12 compares the amplitude of the myoelectric signal of a series of muscle action potentials detected from one or more of the myoelectric detection electrodes 2a, 2b, 2c with the amplitude before the training mode. Then, the muscle condition determination part 12 determines the degree of fatigue of the muscle 50 from an amplitude increase state, and outputs such a determination result to the controller 3. Note that the relative positions of the myoelectric detection electrodes 2a, 2b, 2c closely contacting the vicinity of the muscle 50 have been already provided, and therefore, the degree of fatigue of each portion of the muscle 50 can be more accurately determined from these relative positions and a change in the amplitude of the myoelectric signal shown at each of the myoelectric detection electrodes 2a, 2b, 2c.

The controller 3 displays, on the display 19, the determination result indicating the degree of fatigue of the muscle 50 to notify the user of the determination result. Moreover, when the determination result shows that fatigue of the muscle 50 does not reach a target value in training, the controller 3 further switches the muscle condition determination mode to the training mode to repeat electrical stimulation to the above-described muscle 50. When the degree of fatigue of the muscle 50 reaches the target value in training, the controller 3 displays such a state on the display 19 to terminate operation of the muscle training device 1.

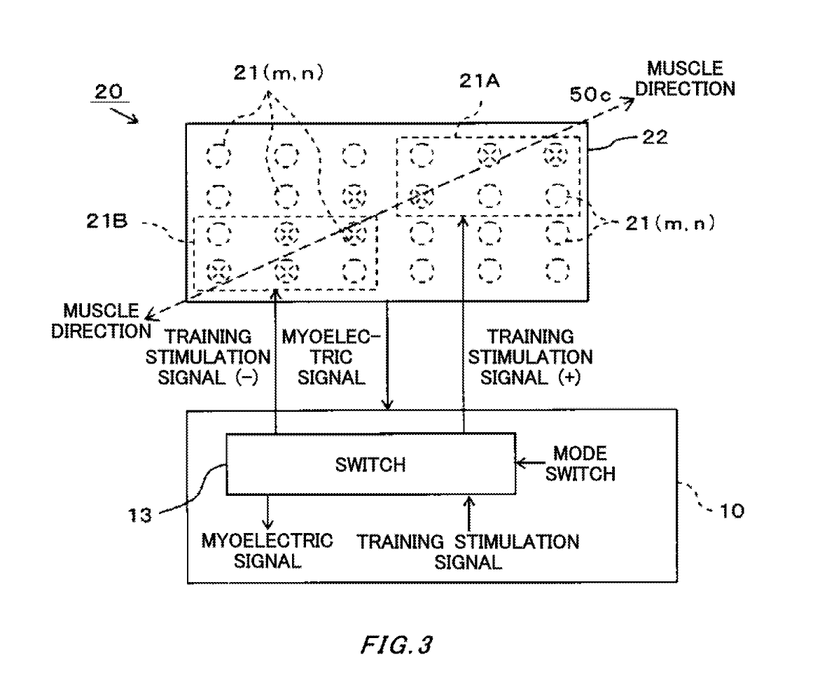

A muscle training device 20 of a second embodiment of the present invention is configured such that a plurality of electrodes 21 are dispersedly exposed on a two-dimensional plane of a bottom surface of an insulating sheet 22, and will be described below with reference to FIG. 3. The same reference numerals as those of the above-described muscle training device 1 are used to represent equivalent elements of the muscle training device 20 functioning in a manner identical or similar to that of the muscle training device 1, and description thereof will not be repeated.

The insulating sheet 22 used for the muscle training device 20 has a horizontally-elongated rectangular profile as viewed in the figure, and the plurality of electrodes 21 (m, n) are dispersedly attached to the insulating sheet 22 in the state in which these electrodes are exposed and positioned in a matrix of four columns and six rows on the bottom surface of the insulating sheet 22. The same number of common terminals and the same number of pairs of first and second selection terminals 13a, 13b switchably connected to a corresponding one of the common terminals as the number of electrodes 21 (m, n) attached to the insulating sheet 22, i.e., 24 common terminals and 24 pairs of first and second selection terminals 13a, 13b, are arranged at a switch 13 of a training device body 10, and each common terminal is connected to a corresponding one of the electrodes 21 (m, n) via a connection cable 7.

In the case where a muscle 50 is trained by the muscle training device 20, the insulating sheet 22 is first bonded to a body surface near the muscle 50. Then, a user operates an operation part 18 to start the device 20 and to voluntarily expand and contract the muscle 50 to be trained. Right after start-up of the muscle training device 20, a controller 3 operates in a muscle condition determination mode to output, to the switch 13, a mode switch signal for selecting all of the electrodes 21 (m, n) to make transition to the muscle condition determination mode. As a result, all of the electrodes 21 (m, n) serve as myoelectric detection electrodes 21, and muscle action potentials detected from all of the electrodes 21 (m, n) are serially input to a muscle condition determination part 12.

When each muscle action potential detected from ones of the electrodes 21 (m, n) indicated by cross marks in FIG. 3 is a relatively-higher muscle action potential as compared to those of the other ones of the electrodes 21 (m, n), the muscle condition determination part 12 estimates that the electrodes 21 (m, n) indicated by the cross marks closely contact the body surface near the muscle 50, and determines that the muscle 50 is present in a muscle direction 50c illustrated in the figure. Then, the muscle condition determination part 12 outputs such a determination result to the controller 3.

After having received the determination result of the position of the muscle 50 from the muscle condition determination part 12, the controller 3 transitions to a training mode. Then, the controller 3 outputs, to the switch 13, a mode switch signal for selecting each electrode 21 (m, n) (m=1 to 2, n=4 to 6) surrounded by a dashed line 21A in the figure as a positive stimulation electrode and selecting each electrode 21 (m, n) (m=3 to 4, n=1 to 3) surrounded by a dashed line 21B in the figure as a negative stimulation electrode to make transition to the training mode. Accordingly, a training stimulation signal flows along the muscle direction 50c indicated by the determination result. Thus, the training stimulation signal for expanding and contracting the muscle 50 flows, from an electrical stimulation output part 14, between the group of the six positive electrodes 21 (m, n) surrounded by the dashed line 21A in the figure and the group of the six negative electrodes 21 (m, n) surrounded by the dashed line 21B in the figure.

As in the first embodiment, for checking the state of fatigue of the muscle 50 due to training in the process of the training mode, the controller 3 is temporarily switched to the muscle condition determination mode so that the muscle action potentials of the electrodes 21 (m, n) serving as the myoelectric detection electrodes can be detected. Then, the muscle condition determination part 12 determines the state of fatigue of the muscle 50 based on comparison of the amplitude of a myoelectric signal of a series of muscle action potentials with the amplitude right after start-up. At this point, connection of the switch 13 is switchably connected to each second selection terminal 13b, and the electrodes 21 (m, n) serving as the myoelectric detection electrodes, such as the electrodes 21 (m, n) surrounded by the dashed line 21A, 21B in the figure or the electrodes 21 (m, n) indicated by the cross marks, can be optionally selected from all of the 24 electrodes 21 (m, n).

As described above, the myoelectric detection electrodes and the stimulation electrodes are not necessarily the same myoelectric detection electrodes 21 (m, n), and can be optionally selected from all of the electrodes 21 (m, n) according to applications or purposes.

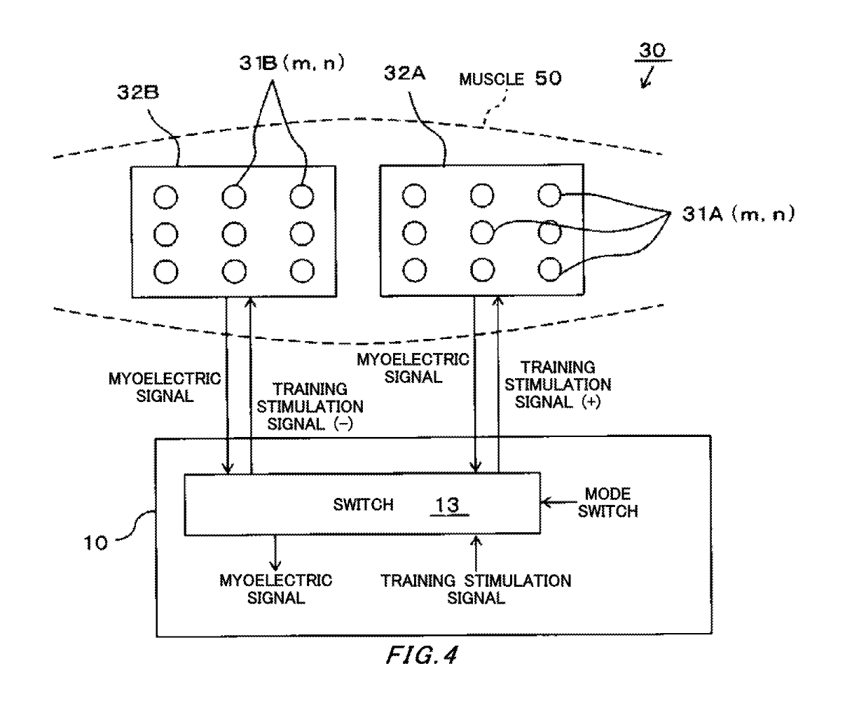

In the case where the entirety of the muscle 50 to be trained cannot be covered with the single insulating sheet 22, a plurality of insulating sheets are bonded to different positions of the body surface above the muscle 50 so that the muscle 50 can be trained by application of the training stimulation signal across a large area. A muscle training device 30 of a third embodiment using two insulating sheets 32A, 32B will be described below with reference to FIG. 4. Note that the same reference numerals as those of the above-described muscle training devices 1, 20 are used to represent equivalent elements of the muscle training device 30 functioning in a manner identical or similar to those of the muscle training devices 1, 20, and description thereof will not be repeated.

As illustrated in FIG. 4, the muscle training device 30 includes the two insulating sheets 32A, 32B, i.e., the insulating sheet 32A on which nine electrodes 31A (m, n) are dispersedly exposed and positioned in a matrix of three columns and three rows on a bottom surface, and the insulating sheet 32B on which nine electrodes 31B (m, n) are dispersedly exposed and positioned in a matrix of three columns and three rows on a bottom surface. Each of the electrodes 31A (m, n) and the electrodes 31B (m, n) is connected to a corresponding one of common terminals of a switch 13 via a connection cable 7, and is selectively connectable to any of a first selection terminal 13a and a second selection terminal 13b. Each of these electrodes can serve as a myoelectric detection electrode and a stimulation electrode according to a mode switch signal output from a controller 3 to the switch 13.

In the present embodiment, the controller 3 operates in a training mode right after the muscle training device 30 has been started. The mode switch signal for selecting all of the electrodes 31A (m, n) exposed on the insulating sheet 32A as positive stimulation electrodes and selecting all of the electrodes 31B (m, n) exposed on the insulating sheet 32B as negative stimulation electrodes to make transition to the training mode is output to the switch 13 such that a training stimulation signal flows across a large area of a muscle 50. Accordingly, the training stimulation signal for extending/contacting the muscle 50 flows, from an electrical stimulation output part 14, between the group of the nine positive stimulation electrodes 31A (m, n) exposed on the insulating sheet 32A and the group of the nine negative stimulation electrodes 31B (m, n) exposed on the insulating sheet 32B. Thus, an electrical stimulation can be, for training, applied to a large area of the muscle 50 which cannot be covered with a single insulating sheet 32.

In the process of the training mode, the controller 3 may be switched to a muscle condition determination mode, and some electrodes 31 of the stimulation electrodes 31A (m, n), 31B (m, n) may serve as the myoelectric detection electrodes. The amplitude of each myoelectric signal detected from these myoelectric detection electrodes may be monitored, and the degree of fatigue of the muscle 50 may be determined from a change in the amplitude of the myoelectric signal.

In each of the above-described embodiments, when the user voluntarily expands and contracts the skeletal muscle 50, the position of the muscle 50 and the degree of fatigue of the muscle 50 are, in the muscle condition determination mode, determined from the level of the muscle action potential generated at the muscle 50 and the amplitude of the myoelectric signal of a series of muscle action potentials. Thus, the muscle 50 to be trained is limited to the skeletal muscle, and the user needs to voluntarily expand and contract the muscle 50 every time the state of action of the muscle 50 is evaluated. For this reason, a muscle stimulation induction signal different from the training stimulation signal may be output to the vicinity of the body surface closely contacting the myoelectric detection electrode, and an induced muscle action potential generated at the muscle 50 by an electrical stimulation of the muscle stimulation induction signal may be detected by the myoelectric detection electrode. Then, the state of action of the muscle 50 may be evaluated from the waveform (the M-wave) of the induced muscle action potential.

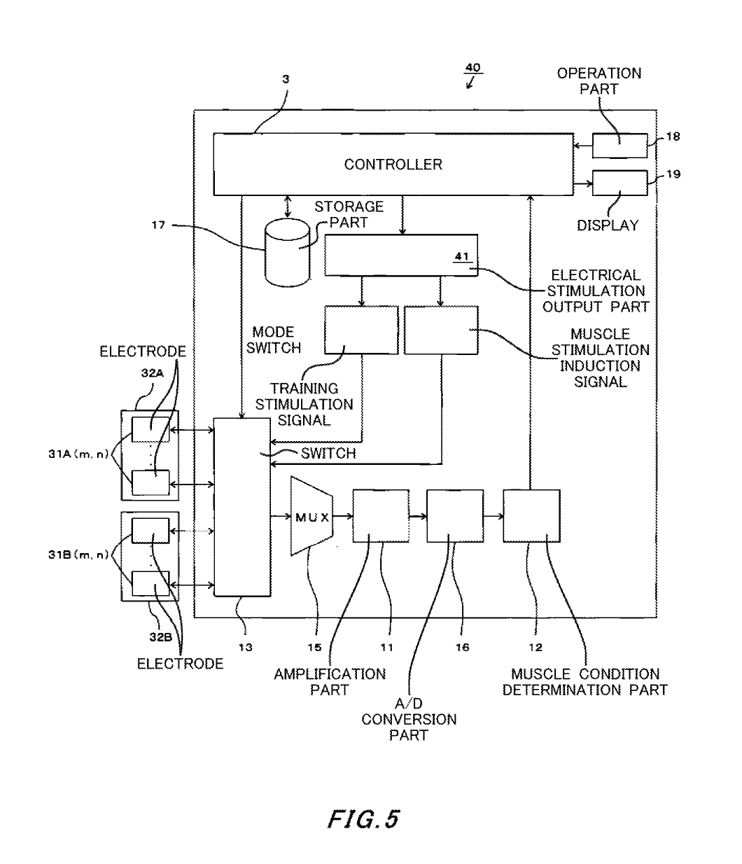

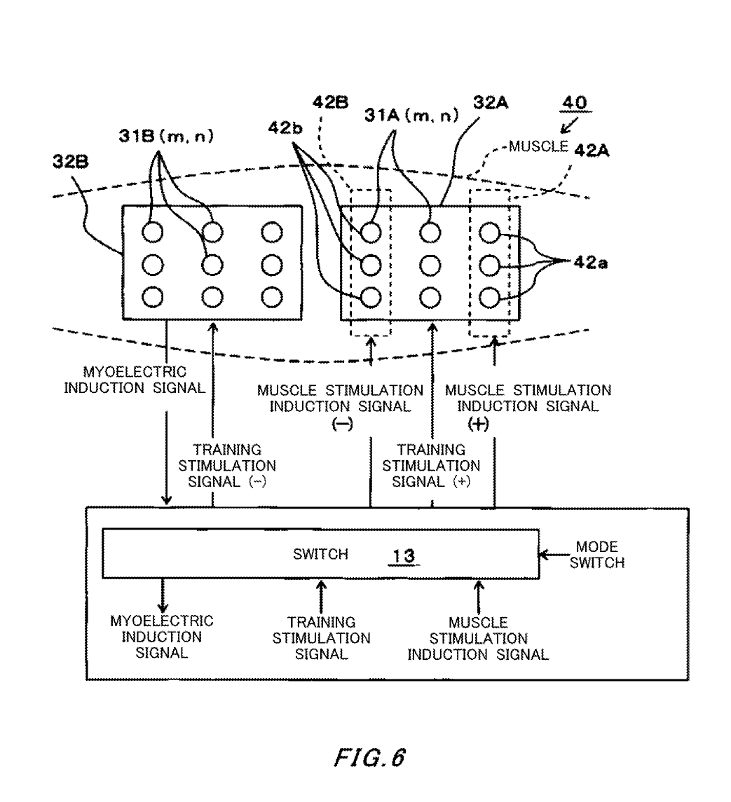

A muscle training device 40 configured to output a muscle stimulation induction signal in a muscle condition determination mode according to a fourth embodiment of the present invention will be described below with reference to FIGS. 5 and 6. The same reference numerals as those of the above-described muscle training devices 1, 20, 30 are used to represent equivalent elements of the muscle training device 40 functioning in a manner identical or similar to those of the muscle training devices 1, 20, 30, and description thereof will not be repeated.

An electrical stimulation output part 41 of the muscle training device 40 is configured to generate two types of electrical stimulation signals, i.e., a training stimulation signal and a muscle stimulation induction signal. The electrical stimulation output part 41 outputs the training stimulation signal to a first selection terminal 13a of a switch 13 when a controller 3 operates in a training mode, and outputs the muscle stimulation induction signal to the first selection terminal 13a of the switch 13 when the controller 3 operates in a muscle condition determination mode. The training stimulation signal generated in the electrical stimulation output part 41 is an electrical stimulation signal having an AC signal waveform with a current value of about 10 to 80 mA and a frequency of 10 to 30 Hz as in the training stimulation signal output from the electrical stimulation output part 14. Moreover, the muscle stimulation induction signal generated in the electrical stimulation output part 41 to generate an induced muscle action potential is an electrical stimulation signal having a rectangular wave with a current value of equal to or greater than 5 mA, a voltage of 100 V, and a pulse width of 0.5 msec, for example.

After start-up of the muscle training device 40, the controller 3 first operates in the training mode to control output of the training stimulation signal from the electrical stimulation output part 41. Moreover, a mode switch signal for selecting all of electrodes 31A (m, n) exposed on an insulating sheet 32A as positive stimulation electrodes and selecting all of electrodes 31B (m, n) exposed on an insulating sheet 32B as negative stimulation electrodes to make transition to the training mode is output to the switch 13 such that the training stimulation signal flows along a muscle 50. Accordingly, the training stimulation signal for expanding and contracting the muscle 50 for training thereof flows, from the electrical stimulation output part 41, between the group of the positive stimulation electrodes 31A (m, n) exposed on the insulating sheet 32A and the group of the negative stimulation electrodes 31B (m, n) exposed on the insulating sheet 32B.

Subsequently, the controller 3 is switched to the muscle condition determination mode at a constant frequency in the process of the training mode. Then, control is made such that output of the training stimulation signal from the electrical stimulation output part 41 is stopped and that the muscle stimulation induction signal is generated and output. Moreover, a mode switch signal for selecting, as positive stimulation sub-electrodes 42a, three electrodes 31A (m, 3) surrounded by a dashed line 42A of FIG. 6 and exposed in a third row on the insulating sheet 32A, selecting, as negative stimulation sub-electrodes 42b, three electrodes 31A (m, 1) surrounded by a dashed line 42B of the figure and exposed in a first row on the insulating sheet 32A, and selecting, as myoelectric detection electrodes, all of the nine electrodes 31B (m, n) exposed on the insulating sheet 32B to make transition to the muscle condition determination mode is output from the controller 3 to the switch 13. Accordingly, each common terminal connected to a corresponding one of the electrodes 31A (m, 3) surrounded by the dashed line 42A is switchably connected to a first selection terminal 13a to which a training stimulation signal (+) is output, each common terminal connected to a corresponding one of the electrodes 31A (m, 1) surrounded by the dashed line 42B is switchably connected to a first selection terminal 13a to which a training stimulation signal (−) is output, and all of the electrodes 31B (m, n) exposed on the insulating sheet 32B are switchably connected respectively to second selection terminals 13b connected to an input of a multiplexer 15.

The negative stimulation sub-electrode 42b and the positive stimulation sub-electrode 42a in a pair are exposed respectively on the right and left sides of the elongated band-shaped insulating sheet 32A. The positive electrodes 42a closely contact a body surface on an outer side with respect to the myoelectric detection electrodes 31B (m, n) exposed on the insulating sheet 32B, and the negative electrodes 42b closely contact the body surface on an inner side with respect to the positive electrodes 42a. When the muscle stimulation induction signal flows between the group of the positive electrodes 42a closely contacting the body surface above the muscle 50 and the group of the negative electrodes 42b closely contacting the body surface above the muscle 50, such a muscle stimulation induction signal flows, during application thereof, inward through the positive electrodes 42a, flows in a longitudinal direction in nerve fibers, and flows outward in the negative electrodes 42b. Outward current induces excitation, and therefore, it is typically considered that the induced muscle action potential is generated in the vicinity of the negative electrodes 42b when signal application begins. Subsequently, the induced muscle action potential is also generated in the vicinity of the positive electrodes 42a. However, since the muscle stimulation induction signal is a short stimulation with 0.5 msec as described above, only the negative electrodes 42b has a stimulation effect when signal application begins. Subsequently, the induced muscle action potential is, along the direction of muscle fibers of the muscle 50, transmitted from the closely-contact positions of the negative electrodes 42b to the myoelectric detection electrodes 31B (m, n) exposed on the insulating sheet 32B.

The waveform of such an induced muscle action potential is the waveform of a combination of waves called “M-waves” and indicating electric action of many muscle fibers of the muscle 50 to be evaluated. A muscle condition determination part 12 monitors the M-wave detected at each of the myoelectric detection electrodes 31B (m, n), and obtains, every time the controller 3 transitions to the muscle condition determination mode in the process of the training mode, a time (a latent time) after application of the muscle stimulation induction signal to the pair of stimulation sub-electrodes 42a, 42b until detection of rising of the M-wave in each of the myoelectric detection electrodes 31B (m, n). The latent time may be obtained for any of the myoelectric detection electrodes 31B (m, n), or may be obtained from an average for the plurality of myoelectric detection electrodes 31B (m, n).

The transmission velocity of the M-wave of the muscle action potential induced by an electrical stimulation decreases with an increase in fatigue of the muscle 50 due to training. On the other hand, the transmission velocity of the M-wave is in inverse proportion to the latent time because a distance from an electrical stimulation position (the stimulation sub-electrode 42b) to each of the electrodes 31B (m, n) is constant. Thus, the muscle condition determination part 12 compares the latent time obtained every time the controller 3 transitions to the muscle condition determination mode, thereby evaluating the degree of fatigue of the muscle 50.

The controller 3 displays, on a display 19, an evaluation result of the degree of fatigue of the muscle 50, the evaluation result being input from the muscle condition determination part 12 every time the controller 3 transitions to the muscle condition determination mode. Moreover, when the degree of fatigue of the muscle 50 reaches a certain value, the controller 3 stops subsequent operation in the training mode to terminate training using the muscle training device 40.

According to the muscle training device 40, the electrical stimulation is, at a constant frequency, applied from each pair of stimulation sub-electrodes 42a, 42b to the muscle 50 during training so that the induced muscle action potential induced by the electrical stimulation can be, even during training, detected distinctively from an unstable muscle action potential generated by a nerve stimulation from the brain. Moreover, the muscle 50 does not need to be consciously expanded and contracted every time the state of action of the muscle 50 is determined. Further, even when the muscle 50 is other muscles than a skeletal muscle, the state of action of the muscle 50 can be determined from the induced muscle action potential shown at each of the myoelectric detection electrodes 31B (m, n).

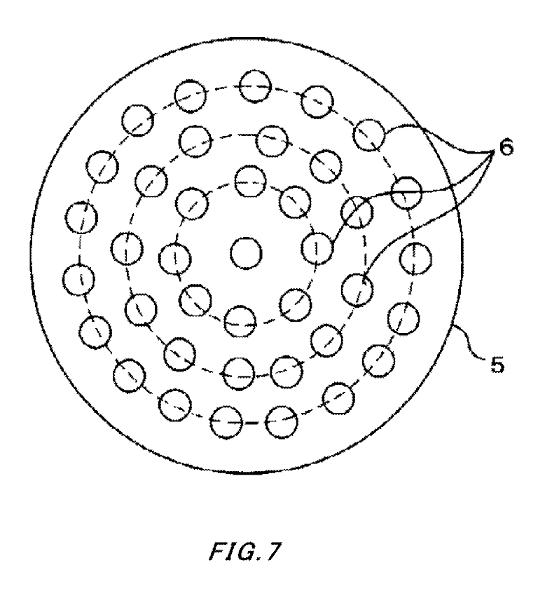

In each of the above-described embodiments, the plurality of electrodes are dispersedly exposed at the linearly-arranged positions of the bottom surface of the insulating sheet or the positions in a matrix on the bottom surface of the insulating sheet. However, the profile of the insulating sheet and the positions of the plurality of exposed electrodes can be optionally set. For example, in the case where a plurality of electrodes are dispersedly exposed on a two-dimensional plane of a bottom surface of an insulating sheet, the insulating sheet 5 may be in a discoid shape, and the plurality of electrodes 6 may be, on the bottom surface of the insulating sheet 5, dispersedly exposed in a concentric pattern at positions on a plurality of circles concentric with the center of the discoid shape, as illustrated in FIG. 7.

According to the above-described insulating sheet 5, the plurality of electrodes 6 are dispersedly exposed on the two-dimensional plane of the bottom surface. Thus, even when the body surface near the muscle 50 to be trained is curved, any of the electrodes 6 closely contacts the vicinity of the muscle 50, and a relatively-high muscle action potential is detected from such an electrode 6. As a result, a training stimulation signal can be effectively applied to the muscle 50.

In the above-described embodiments, each electrode attached to the insulating sheet and the training device body 10 are connected together via the connection cable 7. However, a communication interface configured to perform two-way wireless communication may be provided at each of the insulating sheet and the training device body 10 such that wireless connection among the plurality of electrodes and the common terminals of the switch 13 is realized.

Moreover, the bottom surface of the insulating sheet is, with the adhesive layer, bonded to the body surface above the muscle 50. However, as long as the insulating sheet can be at a predetermined position of the body surface, the insulating sheet may be positioned in such a manner that the insulating sheet is, using a band etc., wound around the body surface.

Further, part or the entirety of the configuration of the training device body 10 is wearable with such a configuration being disposed in a device to be attached to the body with a band etc.

The embodiments of the present invention are suitable for a muscle training device configured to train a muscle by application of an electrical stimulation.

1. A muscle training device for applying a first electrical stimulation signal for training to a body surface above a muscle to expand and contract the muscle by the first electrical stimulation signal, comprising:
an insulating sheet configured such that a plurality of electrodes are exposed on an inner surface closely contacting the body surface above the muscle;
a first stimulation signal output unit configured to output the first electrical stimulation signal to a stimulation electrode which is at least any of the plurality of electrodes;
a myoelectric signal detection unit configured to detect, while output of the first electrical stimulation signal is stopped, a muscle action potential shown at a myoelectric detection electrode which is at least any of the plurality of electrodes; and
a muscle condition determination unit configured to determine a state of action of the muscle from the muscle action potential of the myoelectric detection electrode.

2. The muscle training device according to claim 1, wherein
the stimulation electrode to which the first electrical stimulation signal is output and/or a method for outputting the first electrical stimulation signal are/is adjusted according to the state of action of the muscle determined by the muscle condition determination unit.

3. The muscle training device according to claim 1, wherein
the plurality of electrodes dispersedly exposed on a two-dimensional plane along the inner surface of the insulating sheet serve as myoelectric detection electrodes, and
the stimulation electrode to which the first electrical stimulation signal is output is selected from ones of the plurality of myoelectric detection electrodes from which a relatively-high muscle action potential has been detected.

4. The muscle training device according to claim 3, wherein
the plurality of electrodes dispersedly exposed at positions in a grid pattern on the inner surface of the insulating sheet serve as the myoelectric detection electrodes.

5. The muscle training device according to claim 3, wherein
the plurality of electrodes dispersedly exposed at positions in a concentric pattern on the inner surface of the insulating sheet serve as the myoelectric detection electrodes.

6. The muscle training device according to claim 1, wherein
a stimulation sub-electrode including at least one electrode closely contacts the body surface along the muscle in a vicinity of the body surface to which the insulating sheet closely contacts, and
the first electrical stimulation signal output unit causes one of the stimulation electrode or the stimulation sub-electrode on the insulating sheet to serve as a positive electrode and causes the other one of the stimulation electrode or the stimulation sub-electrode on the insulating sheet to serve as a negative electrode, thereby outputting the first electrical stimulation signal to between the positive and negative electrodes.

7. The muscle training device according to claim 6, wherein
a pair of insulating sheets closely contact two body surface portions along the muscle, and
any of a plurality of electrodes exposed on an inner surface of one of the insulating sheets serves as a stimulation sub-electrode.

8. The muscle training device according to claim 1, further comprising:
a second stimulation signal output unit configured to apply, while output of the first electrical stimulation signal is stopped, a second electrical stimulation signal for generating an induced muscle action potential along the muscle in a vicinity of the body surface to which the insulating sheet closely contacts, wherein
the myoelectric signal detection unit detects the induced muscle action potential shown at the myoelectric detection electrode by application of the second electrical stimulation signal.
